(19) United States
(12) Patent Application Publication
(10) Pub. No.: US 2025/0058089 A1
(43) Pub. Date: Feb. 20, 2025

(54) DECOMPRESSION NEEDLE ASSEMBLY

(71) Applicant: North American Rescue, LLC, Greer, SC (US)

(72) Inventors: Scotty Bolleter, San Antonio, TX (US); Brent Bronson, Duncan, SC (US); Matt Cupelli, Greer, SC (US)

(21) Appl. No.: 18/235,113

(22) Filed: Aug. 17, 2023

(51) Int. Cl. A61M 25/06; A61M 25/00

(52) U.S. Cl. CPC A61M 25/065; A61M 25/0097; A61M 2025/0008

(57) ABSTRACT

A decompression needle assembly may include a catheter, a hub body, and a needle. The catheter may extend between a proximal catheter end (PCE) and a tapered distal catheter end (TDCE). The catheter may define a longitudinal opening at the TDCE. The hub body may be disposed on the catheter at the PCE. The needle may extend between a distal needle end (DNE) and a proximal needle end (PNE). The needle may be selectively received through the hub body and the catheter, the needle having a scalpel tip defined at the DNE.

### FIELD OF THE DISCLOSURE

The present subject matter relates generally to a decompression needle assembly with features for treating, for example, pneumothorax or tension pneumothorax, such as by first responders and providers of emergency medicine.

### BACKGROUND OF THE DISCLOSURE

In the field of medical and trauma care for combat and civilian illness and injury, there exists a dedicated focus on mitigating major preventable causes of death. These causes may include compressible hemorrhage, airway obstruction, or tension pneumothorax. One example, tension pneumothorax, is a leading cause of preventable death in the modern clinical landscape for both military and civilian healthcare providers. Tension pneumothorax can result from penetrating chest trauma as seen on the battlefield, motor vehicle collision, or mass shooting, as well as other medical causes, such as spontaneous pneumothorax or various chronic lung disease. Without treatment, tension pneumothorax may rapidly progress to shock and death. Oftentimes, the immediate treatment of choice is needle decompression.

Tension pneumothorax is the progressive build-up of air within a pleural space of a patient. The subsequent building of pressure caused by the air may prevent the lung from inflating or cause the lung to collapse. This progressive build-up of pressure within the pleural space may also push the mediastinum to the opposing hemithorax, which obstructs venous return, and prevents adequate filing of the heart. This condition often leads to cardiopulmonary instability and may result in hypoxia (i.e., low oxygen levels), hypercarbia (i.e., elevated carbon dioxide levels), hypotension (i.e., low blood pressure), or arrest (i.e., cessation of blood flow).

Historically, the management of tension pneumothorax involves emergent chest decompression with needle thoracostomy. However, this treatment is not without risks. Needle thoracostomies have several potential complications that include improper placement of an assembly (e.g., needle and catheter), blockage of the catheter, kinking of the catheter, or injury (e.g., iatrogenic related) to the lung if the distal end of a portion of the assembly contacts the lung. It is further noted that if the catheter is blocked as the air escapes the pleural space, and the lung is re-inflating, tension may re-accumulate resulting in the clinical problem that was initially being addressed. Still further, existing methods typically require fine motor skills or thought processes to perform the operational procedures. Thus, they may be unsuitable for minimally experienced providers or a dynamic/austere setting in that they are not designed to account for the provider's ability to manage stress. In the dynamic/austere/emergent setting, the provider can be significantly affected by high stress, reaction to a violent situation, and other clinical conditions. The reaction to these conditions can results in tunnel vision, auditory exclusion, the loss of fine and complex motor control, irrational behavior, and the inability to process solutions clearly.

In order to minimize one or more of the above complications, it would be advantageous to have a decompression needle and catheter assembly—or methods of using a decompression needle and catheter assembly—that has improved insertion characteristics, reduces or eliminates blockage, and limits or prevents kinking of the catheter or damage to the lung. Additionally or alternatively, it may be advantageous to have a decompression needle and catheter assembly—or methods of using a decompression needle and catheter assembly—that prevents pressure in the pleural space could also drain fluid from within the pleural space through the catheter. Further additionally or alternatively, it may be advantageous to have a decompression needle and catheter assembly—or methods of using a decompression needle and catheter assembly—that is suitable for a dynamic/austere/emergent setting in which fine motor skills, thought processes, or operational procedures are difficult to maintain. For example, in the combat or civilian prehospital setting having a decompression needle that can be carried by each person engaged would allow the provider to use the injured person's supplies without having to carry multiple devices.

### BRIEF DESCRIPTION OF THE DISCLOSURE

Aspects and advantages of the invention will be set forth in part in the following description, or may be obvious from the description, or may be learned through practice of the invention.

In one exemplary aspect of the present disclosure, a decompression needle assembly is provided. The decompression needle assembly may include a catheter, a hub body, and a needle. The catheter may extend between a proximal catheter end (PCE) and a tapered distal catheter end (TDCE). The catheter may define a longitudinal opening at the TDCE. The hub body may be disposed on the catheter at the PCE. The needle may extend between a distal needle end (DNE) and a proximal needle end (PNE). The needle may be selectively received through the hub body and the catheter, the needle having a scalpel tip defined at the DNE.

In another exemplary aspect of the present disclosure, a method of operating a decompression needle assembly is provided. The method may include driving the decompression needle assembly along an axial direction within a patient toward a pleural space such that a distal needle end of a needle contacts a middle portion of a rib. The method may also include noting a proximal marking of a plurality of markings defined on a catheter. The proximal marking may be most proximal to an epidermis surface of the patient and outside thereof. The method may further include selecting a distal marking of the plurality of markings. The distal marking may be a set number rearward from the proximal marking. The method may still further include pivoting the needle within a set range from the axial direction within the patient and further driving the decompression needle assembly at the set range within the pleural space to the selected marking.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a decompression catheter assembly according to exemplary embodiments of the present disclosure.

FIG. 2 provides a side perspective view of the exemplary decompression catheter assembly of FIG. 1.

FIG. 3 provides a perspective view of a portion of a decompression catheter assembly according to exemplary embodiments of the present disclosure, wherein a needle has been partially separated from a needle hub.

FIG. 4 provides a cross-sectional perspective view of a portion of a method of using a decompression catheter assembly according to exemplary embodiments of the present disclosure.

FIG. 5 provides a cross-sectional perspective view of a portion of a method of using a decompression catheter assembly according to exemplary embodiments of the present disclosure.

FIG. 6 provides a cross-sectional perspective view of a portion of a method of using a decompression catheter assembly according to exemplary embodiments of the present disclosure.

FIG. 7 provides a cross-sectional perspective view of a portion of a method of using a decompression catheter assembly according to exemplary embodiments of the present disclosure.

FIG. 8 provides a cross-sectional perspective view of a portion of a method of using a decompression catheter assembly according to exemplary embodiments of the present disclosure.

FIG. 9 provides a cross-sectional perspective view of a portion of a method of using a decompression catheter assembly according to exemplary embodiments of the present disclosure.

FIG. 10 provides a side perspective view of a portion of a decompression catheter assembly according to exemplary embodiments of the present disclosure.

FIG. 11 provides a top perspective view of a needle of a decompression catheter assembly according to exemplary embodiments of the present disclosure.

FIG. 12 provides a side perspective view of a needle of a decompression catheter assembly according to exemplary embodiments of the present disclosure.

FIG. 13 provides a top perspective view of a needle of a decompression catheter assembly according to exemplary embodiments of the present disclosure.

FIG. 14 provides a side perspective view of a needle of a decompression catheter assembly according to exemplary embodiments of the present disclosure.

FIG. 15 provides a front perspective view of a needle of a decompression catheter assembly according to exemplary embodiments of the present disclosure.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components or systems. For example, the approximating language may refer to being within a 10 percent margin (i.e., including values within ten percent greater or less than the stated value). In this regard, for example, when used in the context of an angle or direction, such terms include within ten degrees greater or less than the stated angle or direction (e.g., “generally vertical” includes forming an angle of up to ten degrees in any direction, such as, clockwise or counterclockwise, with the vertical direction V).

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

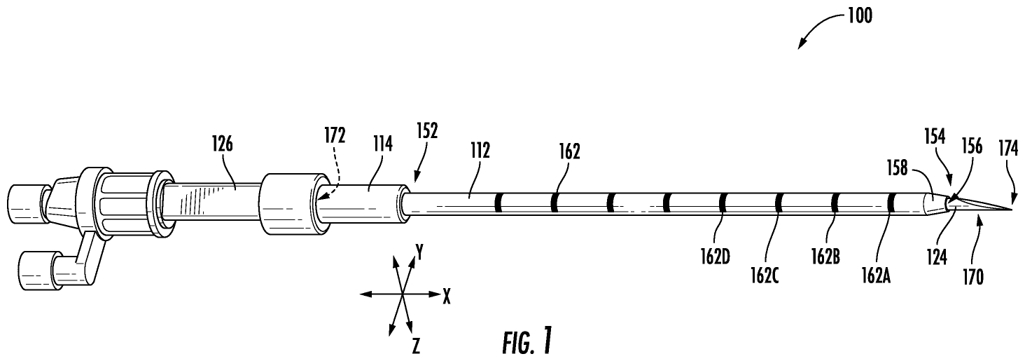

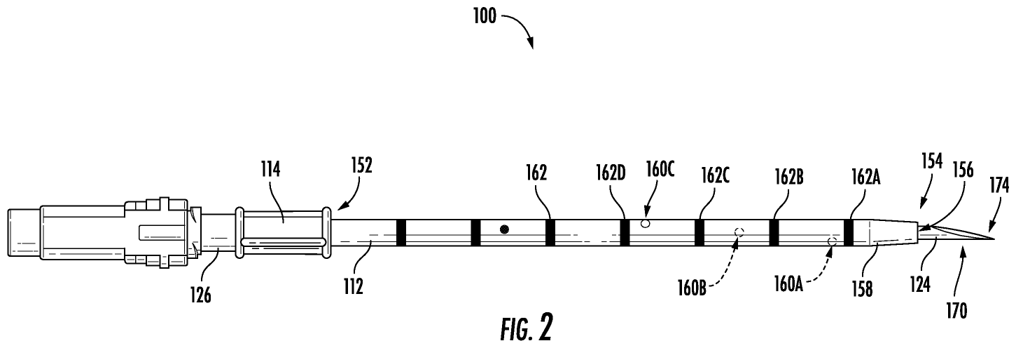

Referring now to the figures, FIGS. 1 and 2 provide various perspective views of an assembled decompression needle assembly 100 according to exemplary embodiments of the present disclosure. As shown, the decompression needle assembly 100 includes a catheter 112. Generally, the catheter 112 extends (e.g., longitudinally) between a proximal catheter end (PCE) 152 and a tapered distal catheter end (TDCE) 154. At the TDCE 154, the catheter 112 defines a longitudinal opening 156. In turn, the catheter 112 may have an outward flow path defined from the TDCE 154 to the PCE 152. The PCE 152 may be attached to a hub body 114. Thus, the hub body 114 may be disposed on the catheter 112 at the PCE 152. Opposite from the hub body 114 (e.g., at the TDCE 154) a resilient tapered tip 158 that is formed from a softer or relatively deformable material from the rest of catheter 112 may be provided. Although formed from a resilient material, resilient tapered tip 158 may be configured to maintain a set shape and resist bending (e.g., curvature). Specifically, the resilient tapered tip 158 of catheter 112 may define longitudinal opening 156 and notably prevent puncture of soft tissue during contact of the same with the catheter 112.

Separate from or in addition to the longitudinal opening 156, catheter 112 may define one or more lateral fenestrations (e.g., a plurality of fenestrations or openings 160A, 160B, 160C) in fluid communication (e.g., upstream fluid communication) with the flow path. As shown, such lateral fenestrations 160A, 160B, 160C may be disposed rearward from the longitudinal opening 156 between the PCE 152 and the TDCE 154. Nonetheless the lateral fenestrations 160A, 160B, 160C may each be disposed proximal to the TDCE 154 (i.e., relatively distal or further from the PCE 152). Moreover, the lateral fenestrations 160A, 160B, 160C may be longitudinally spaced apart (i.e., from each other) or disposed rearward from the resilient tapered tip 158 (i.e., such that the resilient tapered tip 158 is disposed at the TDCE 154 and forward from the plurality of lateral fenestrations 160A, 160B, 160C). Notably, the lateral fenestrations 160A, 160B, 160C may allow fluid to travel along the flow path even if the longitudinal opening 156 or one or more of the fenestrations 160A, 160B, 160C is obstructed.

In some embodiments, multiple lateral fenestrations 160A, 160B, 160C are circumferentially spaced apart (e.g., about a longitudinal direction or axis extending through the catheter 112 between the PCE 152 and the TDCE 154). For instance, a predefined offset angle θ (FIG. 15) may be defined between circumferentially adjacent lateral fenestrations, 160B, 160C (e.g., 160A-160B or 160B-160C). Optionally, multiple adjacent pairs may be spaced apart by the same predefined offset angle θ.

In additional or alternative embodiments, multiple lateral fenestrations 160A, 160B, 160C are longitudinally spaced apart (e.g., along a longitudinal direction or axis extending through the catheter 112 between the PCE 152 and the TDCE 154). For instance, a predefined longitudinal spacing may be defined between longitudinally adjacent lateral fenestrations 160A, 160B, 160C (e.g., 160A-160B or 160B-160C). Optionally, multiple adjacent pairs may be spaced apart by the same longitudinal spacing (e.g., by the same distance or magnitude along the longitudinal direction).

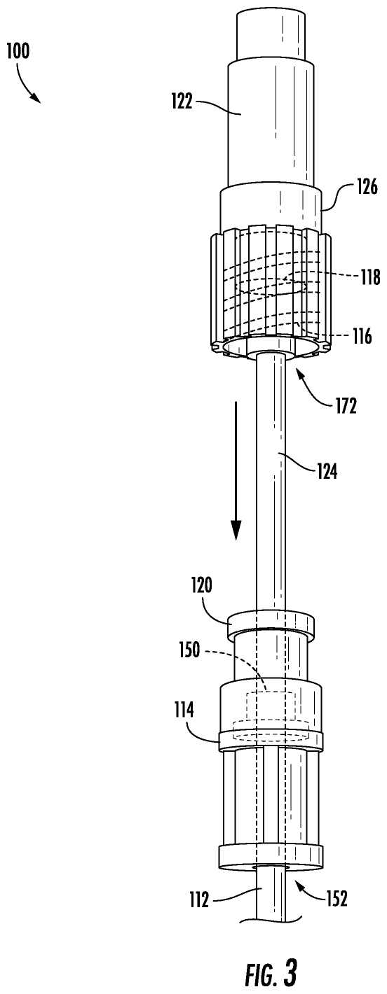

In certain embodiments, a plurality of distance markers 162 are included with the catheter 112. Specifically, the distance markers 162 may be formed or printed on an outer surface of the catheter 112 (e.g., to be visible by a user 202—FIGS. 4 through 9). Optionally, such distance markers 162 may each be shaped, embedded, formed, or provided as hashes or circumferential lines (e.g., circles disposed about a longitudinal direction or axis extending through the catheter 112 between the PCE 152 and the TDCE 154). As shown, the distance markers 162 may be longitudinally spaced apart (e.g., along a longitudinal direction or axis extending through the catheter 112 between the PCE 152 and the TDCE 154). For instance, a predefined longitudinal spacing may be defined between longitudinally adjacent distance pair (e.g., 162A-162B or 162B-162C or 162C-162D). Optionally, multiple adjacent pairs may be spaced apart by the same longitudinal spacing (e.g., by the same distance or magnitude along the longitudinal direction). Thus, the distance markers 162 may be spaced apart from each other. In some embodiments wherein lateral fenestrations 160A, 160B, 160C are provided, each lateral fenestration 160A, 160B, or 160C may be longitudinally bookended by a discrete pair of lateral markers 162 of the plurality of distance markers 162. Thus, on opposite longitudinal sides of each fenestration 160A, 160B, or 160C, a distance marker 162 may be provided, thereby longitudinally bounding each lateral fenestration 160A, 160B, or 160C between two discrete distance markers 162.

In optional embodiments, apart from any distance markers 162 or fenestrations 160A, 160B, 160C, the rear portion of the hub body 114 for catheter 112 includes one or more lock tabs 120 (FIG. 3) that each extends laterally from the hub body 114 (e.g., perpendicular to the outward flow path). A one-way valve 150 may be disposed (e.g., pivotably disposed) within the hub body 114 and configured to open when a set pressure is applied along the flow path and prevents fluid flow in a direction opposite the flow path. It is noted that the one-way valve 150 is held in the open position by the needle 124 when the needle 124 extends through the one-way valve 150. When the needle 124 is removed, the one-way valve 150 closes preventing air/fluid from flowing in the direction of the TDCE 154. The one-way valve 150 can be secured by a valve base that is secured to the one-way valve 150 portion of the hub body 114.

In some embodiments, a needle 124 is provided and extends (e.g., longitudinally) between a proximal needle end (PNE) 172 and a distal needle end (DNE) 174. When assembled (e.g., prior to use), the needle 124 may be selectively received through the hub body 114 and the catheter 112. For instance, the needle 124 can be received in the catheter 112 from the PCE 152. In embodiments including the one-way valve 150, the needle 124 may further pass through the one-way valve 150. Irrespective of the one-way valve 150, the longitudinal length of the needle 124 may be greater than the longitudinal length of the catheter 112. In turn, the assembled needle 124 may extend from the distal end of the catheter 112 (e.g., such that a tip of the needle 124 or PNE 172 is exposed outside of the catheter 112). As will be described in greater detail below, the needle 124 may include a scalpel tip 170 defined at the DNE 174.

Turning especially to FIG. 3, in optional embodiments, a needle hub 126 includes a chamber 122 to assist in indicating when the needle 124 has been properly inserted into a patient 204 (FIGS. 4 through 9). This indication may be auditory (such as a whistle or click) or visual (such as a movement, fluid, or colorimetric indication). A lock collar 118 can be included in the hub body 114 and attached to the forward portion of the hub body 114 adjacent to the catheter 112 when the catheter 112 and needle 124 are assembled. In one embodiment, the temporary lock collar 118 can rotate about needle hub 126 and engage the lock tabs 120 to attach or disarticulate the needle hub 126 to or from the hub body 114. The lock collar 118 can include internal threads 116 that engage or disengage the tabs to secure or release the catheter 112 from the needle 124. In the manufacturer of the assembly 100, the needle 124 can extend through the one-way valve 150.

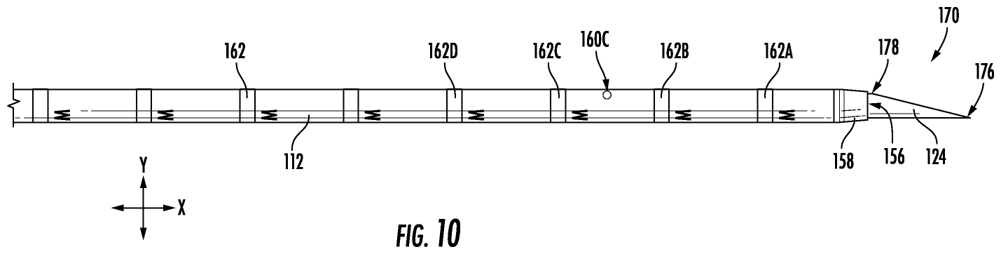

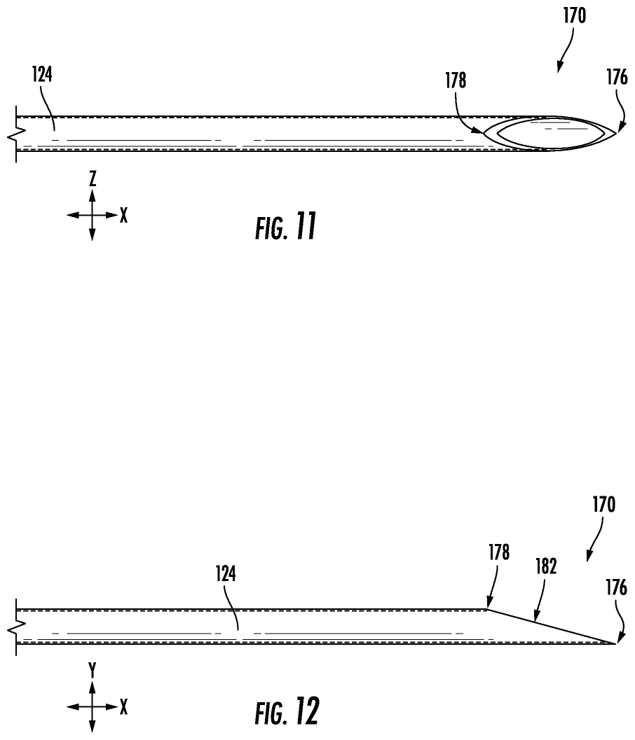

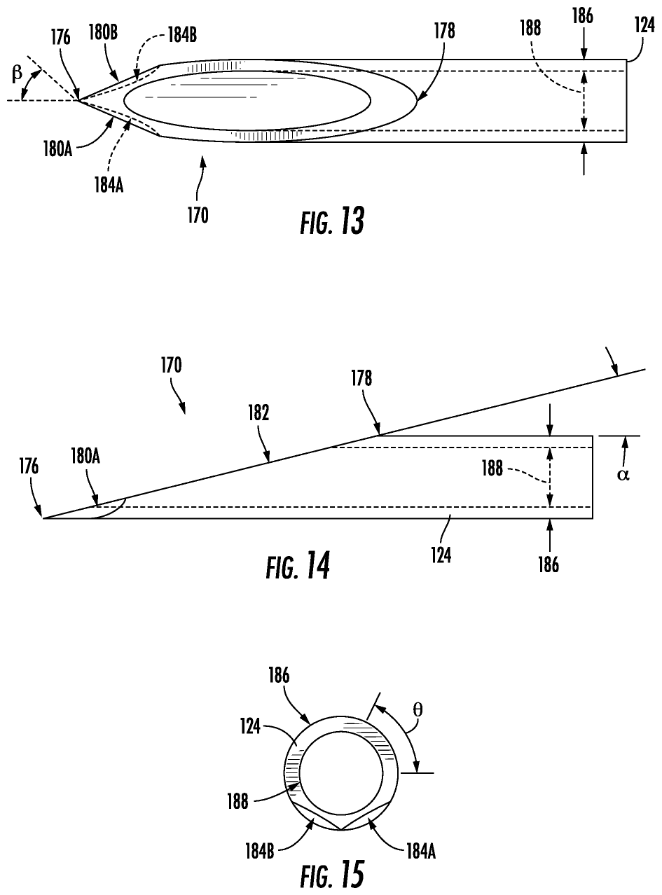

Turning now to FIGS. 10 through 15, various views are provided of the DNE 174 of needle 124, wherein the needle 124 defines a mutually orthogonal X-axis, Y-axis, and Z-axis. As noted above, at the DNE 174, a scalpel tip 170 may be provided. Such as scalpel tip 170 may extend between a forward extremity 176 and a rearward extremity 178 at a set slope angle α (e.g., relative to a X-Z plane, which may be parallel to or extend over rearward extremity 178). In other words, a sloped surface 182 (e.g., specific sloped surface) of the needle 124 may be defined at the set slope angle α between the forward extremity 176 and the rearward extremity 178. In some embodiments, the set slope angle α is between 10° and 20°. For instance, the set slope angle α may be approximately 14°.

In certain embodiments, one or more chiseled edges 180A, 180B are provided at scalpel tip 170. Specifically, the chiseled edge(s) 180A, 180B may extend rearward from forward extremity 176 (e.g., along the sloped surface 182). Furthermore, a chiseled edge 180A or 180B may be formed at a set edge angle β (e.g., relative to a X-Y plane). Optionally, a pair of chiseled edges 180A, 180B may be defined at opposite angles (e.g., of equal magnitude relative to a X-Y plane) from or lateral sides of the forward extremity 176. Thus, an arrowhead or point may be defined at forward extremity 176, as is illustrated. Moreover, for each chiseled edge 180A or 180B, a set edge angle β between the forward extremity 176 and the rearward extremity 178. In some embodiments, the set edge angle β is between 15° and 25°. For instance, the set edge angle β may be approximately 22°. When formed, the chiseled edge 180A or 180B may provide a sharpened cutting surface, as would be understood in light of the present disclosure.

In additional or alternative embodiments, the scalpel tip 170 may include one or more bevel notches 184A, 184B. For instance, a pair of sharpening bevel notches 184A, 184B may be defined at the DNE 174 (e.g., at least partially apart from the set slope angle α, sloped surface 182, chiseled edges 180A, 180B, or set edge angles β). As shown, a pair of bevel notches 184A, 184B may be provided. In some embodiments, the pair of bevel notches 184A, 184B define recesses inward from another portion of the outer surface of the needle 124. As shown, although the bevel notches 184A, 184B descend from an outer surface (e.g., outer diameter 186 of needle 124), the bevel notches 184A, 184B may terminate prior to the inner surface (e.g., inner diameter 188 of needle 124) defining the flow path. The pair of bevel notches 184A, 184B may be defined at opposite lateral sides of the forward extremity 176 (e.g., below the sloped surface 182 or the chiseled edges 180A, 180B). Moreover, each bevel notch 184A or 184B may extend inward or downward from a corresponding chisel edge 180A or 180B.

Notably, the needle 124 and associated scalpel tip 170 may be usable as a cutting utensil (e.g., in contrast to the pure puncture utensil provided by existing needles) capable of cutting a surface portion of a patient's skin (i.e., epidermis and dermis) (e.g., at 212 to prepare the surface for a more accurate subsequent puncture and insertion of the needle 124 and catheter 112). Advantageously, the needle 124 may be able to create an incision without requiring a separate cutting utensil, as might be otherwise required or difficult to provide in a dynamic/austere setting.

Turning now to FIGS. 4 through 9, methods of performing a using a decompression catheter assembly (e.g., decompression needle assembly 100) will be described in greater detail. As would be understood, such methods may be performed by a user 202 operating on a patient 204 while using a decompression needle assembly 100 according to one or more of the above-described embodiments.

Turning especially to FIG. 4, in preparation for relieving tension Pneumothorax in a patient's body cavity or pleural space 206, the fully assembled assembly 100 may be provided with the catheter 112 being received within the needle 124. Outside of the patient 204, the user 202 may hold the assembly 100 and identify a target rib 208 at which the assembly 100 may be driven (e.g., along an axial direction A). In some embodiments, the user 202 may utilize the scalpel tip 170 to cut a small incision above or outward from the rib 208 (e.g., directly above). Thus, the method may include cutting (e.g., prior to puncture or driving the decompression needle assembly 100) a non-puncture incision on the epidermis/dermis surface 212 of the patient 204 outside thereof (i.e., outside of the patient's body).

Turning especially to FIG. 5, the method may subsequently include puncturing the epidermis/dermis surface 212 to insert the needle 124 and catheter 112. Moreover, the method includes driving the decompression needle assembly 100 along the axial direction A within the patient 204 (e.g., through an area below the skin 212—adipose tissue). In some embodiments, driving or movement of the needle 124 and catheter 112 along the axial direction A continues until the DNE 174 contacts a middle portion of the target rib 208 (e.g., a forward-facing surface between a top and a bottom portion of the target rib 208). Following or in response to contacting the target rib 208 (e.g., in a controlled manner), the method may including noting a proximal marker 162A of the plurality of markers 162. As shown, the proximal marker 162A is most proximal to the epidermis/dermis surface 212 of the patient 204 (e.g., outside of the patient 204) and may be noted or selected by the user 202 (e.g., using a front or index finger).

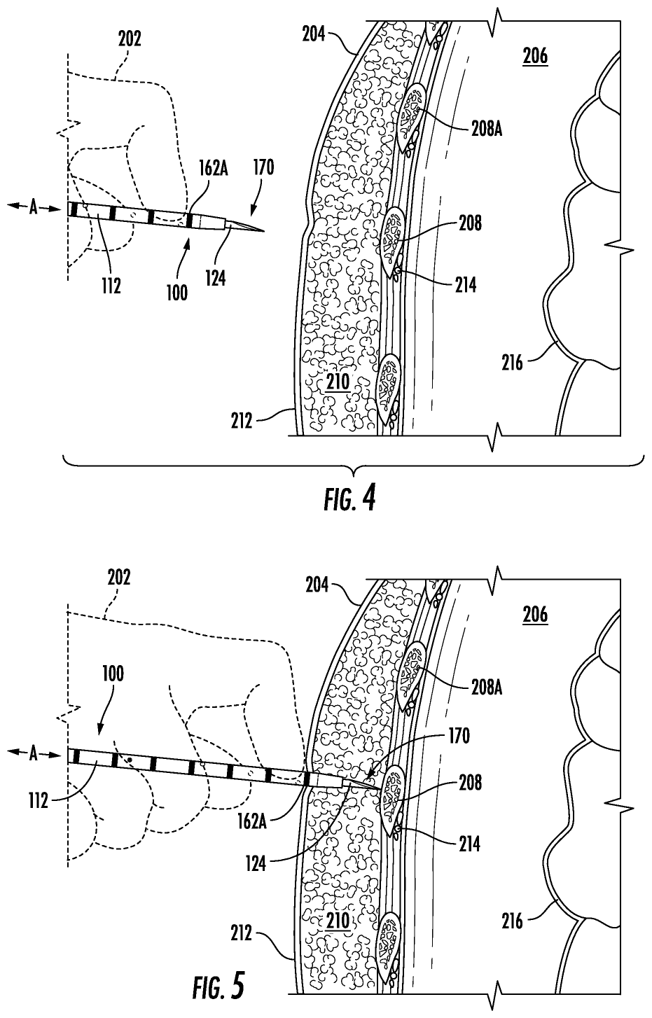

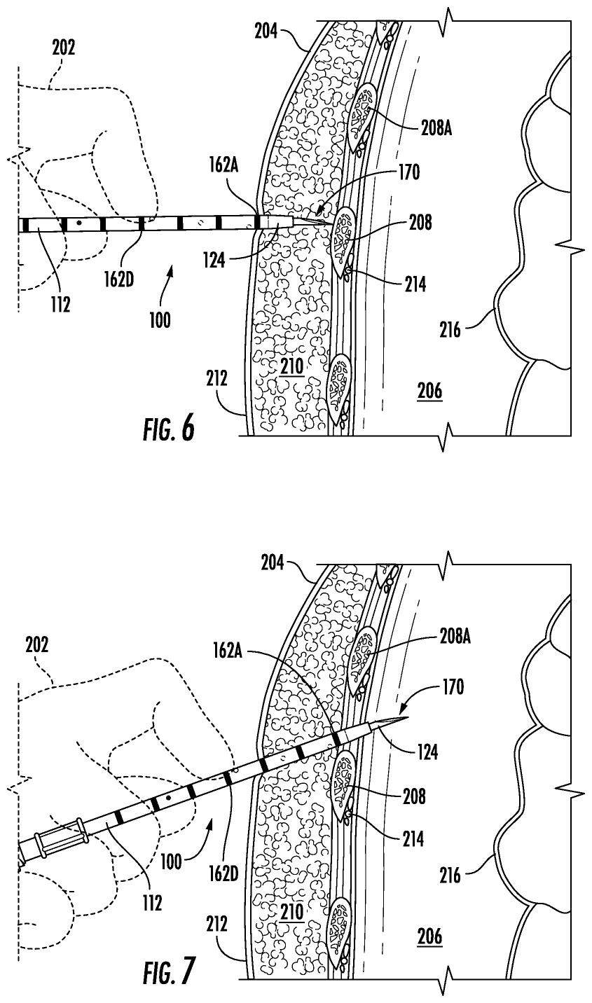

Turning especially to FIG. 6, while the assembly is still in roughly the same position as FIG. 5, the method may further include selecting a distal marker 162D of the plurality of markers 162. For instance, the user 202 may move a finger rearward from the proximal marker 162A to the distal marker 162D. Generally, the distal marker 162D may be a set number of markers rearward from the proximal marker 162A. In the illustrated embodiment, the set number is three, though any suitable predetermined number (e.g., one or more) may be used. Subsequently or prior to further moving or pivoting of the needle 124, the method may include withdrawing the DNE 174 within the patient 204 (e.g., within the area below the skin 212) along the axial direction A (e.g., rearward). In other words, the needle 124 may be moved apart from or out of contact with the target rib 208 (e.g., less than a single longitudinal spacing between markers 162) while keeping the DNE 174 inside of the patient 204.

Turning especially to FIG. 7, the method may further include pivoting the needle 124 within the patient 204 (e.g., subsequent to selecting the distal marker 162D). In particular, the needle 124 may be pivoted within a set range from the axial direction A within the patient 204. For instance, relative to the axial direction A, the needle 124 may be pivoted to an angle between 10° and 45°. The set range may be a positive value such that the needle 124 is pivoted upward over the target rib 208 (e.g., away from nerves and vascular structures 214 disposed at a lower portion of the target rib 208). In turn, the needle 124 may be aimed or directed at a space between adjacent ribs (e.g., between the target rib 208 and an upper rib 208A above the target rib 208). Moreover, the method may subsequently include further driving the decompression needle assembly 100 at the set range within the pleural space 206 to the selected marker 162D. Thus, driving of the needle 124 may cease upon reaching the selected marker 162D. Moreover, the needle 124 and catheter 112 may be held between the target rib 208 and the upper rib 208A (e.g., at least temporarily).

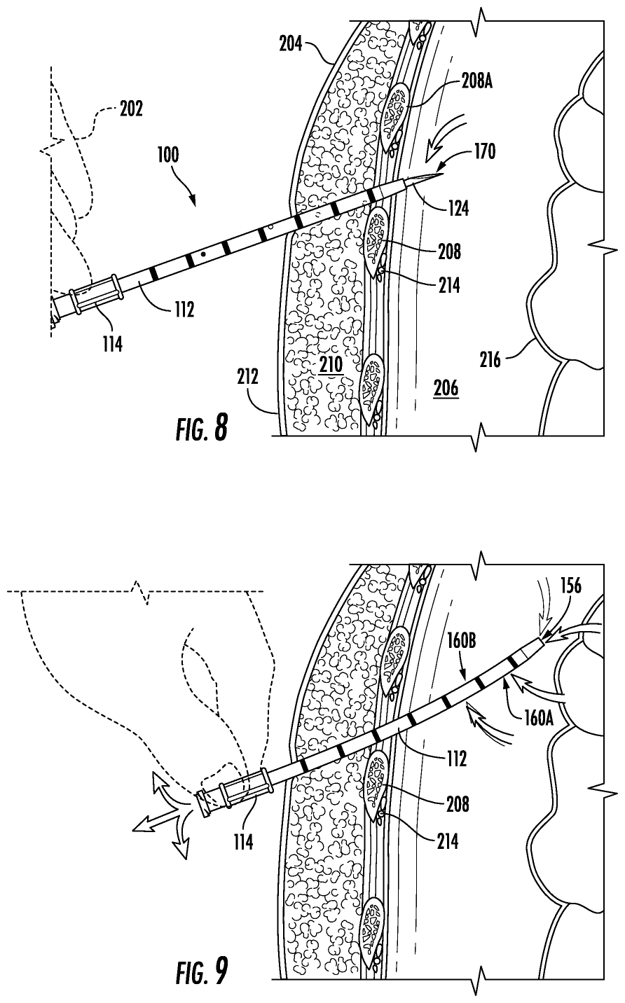

Turning especially to FIG. 8, the method may subsequently (e.g., after reaching the selected marker 162D) include removing the needle 124 from the pleural space 206 while holding the catheter 112 within the pleural space 206. Thus, the needle 124 exits the body of the patient 204 while the catheter 112 remains therein to relieve pressure (e.g., from fluid flowing through the catheter 112 and to a region outside of the patient's body 204).

Turning especially to FIG. 9, the needle 124 may be used as a guide for driving the catheter 112 along the pleural space 206 (e.g., past the selected marker 162D). In some embodiments, the catheter 112 is driven until the hub body 114 reaches the epidermis/dermis surface 212 of the patient 204. As described above, fluid may continue to flow through the catheter 112 via the longitudinal opening 156 or one or more of the fenestrations 160A, 160B, 160C, thereby relieving pressure within the pleural space 206. Moreover, a lung 216 of the patient 204 may be permitted to re-inflate and pressure may be relieved from the heart and vasculature of the patient when the catheter 112 is in the pleural space 206.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A decompression needle assembly comprising:
decompression needle assembly comprising:
a catheter extending between a proximal catheter end (PCE) and a tapered distal catheter end (TDCE), the catheter defining a longitudinal opening at the TDCE;
a hub body disposed on the catheter at the PCE; and
a needle extending between a distal needle end (DNE) and a proximal needle end (PNE), the needle being selectively received through the hub body and the catheter, the needle having a scalpel tip defined at the DNE.

2. The decompression needle assembly of claim 1, wherein the catheter defines a plurality of distance markings longitudinally spaced apart between the PCE and the TDCE.

3. The decompression needle assembly of claim 2, wherein the distance markings are equally spaced apart from each other.

4. The decompression needle assembly of claim 1, wherein the catheter further defines a plurality of lateral fenestrations rearward from the longitudinal opening between the PCE and the TDCE.

5. The decompression needle assembly of claim 4, wherein the plurality of lateral fenestrations are circumferentially and longitudinally spaced apart.

6. The decompression needle assembly of claim 4, wherein the catheter further defines a plurality of a lateral fenestrations between the PCE and the TDCE, and wherein each lateral fenestration of the plurality of lateral fenestrations is longitudinally bookended by a discrete pair of lateral fenestrations of the plurality of lateral fenestrations.

7. The decompression needle assembly of claim 4, wherein the catheter comprises a resilient tapered tip at the TDCE and forward from the plurality of lateral fenestrations.

8. The decompression needle assembly of claim 1, wherein the scalpel tip defines a needle slope at a set slope angle between 10° and 20°.

9. The decompression needle assembly of claim 1, wherein the scalpel tip comprises a pair of sharpening bevel notches at the DNE.

10. A method of using a decompression needle assembly comprising catheter extending between a proximal catheter end (PCE) and a tapered distal catheter end (TDCE), a hub body disposed on the catheter, and a needle extending between a distal needle end (DNE) and a proximal needle end (PNE) and having a scalpel tip defined at the DNE, the method comprising:
driving the decompression needle assembly along an axial direction within a patient toward a pleural space such that the DNE contacts a middle portion of a rib;
noting a proximal marking of a plurality of markings defined on the catheter, the proximal marking being most proximal to an epidermis surface of the patient and outside thereof;
selecting a distal marking of the plurality of markings, the distal marking being a set number rearward from the proximal marking;
pivoting the needle within a set range from the axial direction within the patient; and
further driving the decompression needle assembly at the set range within the pleural space to the selected marking.

11. The method of claim 10, further comprising: cutting, prior to driving the decompression needle assembly along the axial direction, a non-puncture incision on the epidermis surface of the patient.

12. The method of claim 10, wherein the set number is three.

13. The method of claim 10, further comprising: withdrawing, prior to pivoting the needle, the DNE within the patient along the axial direction.

14. The method of claim 10, further comprising: removing the needle from the pleural space while holding the catheter within the pleural space.

15. The method of claim 14, further comprising: driving, following removing the needle, the catheter assuming within the pleural space past the selected distal marking.

16. The method of claim 10, wherein the catheter defines a plurality of distance markings longitudinally spaced apart between the PCE and the TDCE, and wherein the distance markings are equally spaced apart from each other.

17. The method of claim 10, wherein the scalpel tip defines a needle slope at a set slope angle between 10° and 20°.

18. The method of claim 10, wherein the scalpel tip comprises a pair of sharpening bevel notches at the DNE.
